(19) United States
(12) Patent Application Publication
(10) Pub. No.: US 2019/0150704 A1
(43) Pub. Date: May 23, 2019

(54) INSERTION APPARATUS

(71) Applicant: Olympus Corporation, Tokyo (JP)

(72) Inventor: Yasuhiro Okamoto, Tokyo (JP)

(73) Assignee: Olympus Corporation, Tokyo (JP)

(21) Appl. No.: 16/256,428

(22) Filed: Jan. 24, 2019

Related U.S. Application Data
(63) Continuation of application No. PCT/JP2017/008106, filed on Mar. 1, 2017.

(30) Foreign Application Priority Data
Aug. 2, 2016 (JP) 2016-152114

(51) Int. Cl. A61B 1/00; A61B 1/005

(52) U.S. Cl. CPC A61B 1/0016; A61B 1/00066; A61B 1/00133; G02B 23/2476; A61B 1/0051; A61B 1/00121; A61B 1/00154

(57) ABSTRACT

The disclosed technology is directed to an insertion apparatus comprises an insertion portion having a tubular body freely rotates around a longitudinal axis over an outer circumferential surface. The insertion portion is flexible and configured to be inserted into a body cavity. A drive source is configured to rotate the tubular body wherein a part of the insertion portion includes a predetermined flexural rigidity to which the tubular body being mounted thereto. The part of the insertion portion is formed of a structure that is configured in such a manner that bending of the tubular body is not caused beyond a predetermined bending angle so as to avoid stop of rotation of the tubular body by a driving force of the drive source even when an external force that intends to keep a bending shape of the body cavity is received from a wall of the body cavity in contact.

### CROSS-REFERENCE TO RELATED APPLICATIONS

This application is a continuation application of PCT Application No. PCT/JP2017/008106 filed on Mar. 1, 2017, which in turn claim priority to the Japanese Patent Application No. 2016-152114 filed on Aug. 2, 2016 in Japan which is hereby incorporated by reference in its entirety.

### TECHNICAL FIELD

The technology disclosed herein generally relates to insertion apparatus having a drive source disposed in a flexible tube, a driven member, and a transmitting member that is provided in the flexible tube along a long axis which transmits a rotational driving force of the drive source to the driven member.

### DESCRIPTION OF THE RELATED ART

The endoscope is used in the medical field, the industrial field, and so forth. With the endoscope for medical use, observation, examination, procedure, or the like can be carried out by inserting an insertion portion into a body for examination.

Generally, the endoscope has the insertion portion, an operation unit, and a universal cord. In a configuration having a flexible tube part in the insertion portion, the insertion portion is inserted into a digestive organ or digestive tract that is a body cavity transanally, transorally, or transnasally.

The endoscope is configured in such a manner that the flexible tube part of the insertion portion includes a corrugated tube having flexibility. When the insertion portion having the flexible tube part is inserted into an intestinal tract for example, the user inserts the insertion portion located outside the body toward a deep part of the intestinal tract by carrying out twist operation or feed operation while operating a bending operation knob provided in the operation unit to bend a bending part.

However, the twist operation and the feed operation, which is a technique to smoothly insert the insertion portion toward a deep part of a body cavity, requires skillfulness. For this reason, regarding the endoscope, an electric mechanism part such as an insertion support mechanism for causing the insertion portion to advance and retreat toward and from a deep part is disclosed in International Patent Publication No. WO 2015-072233.

Regarding insertion apparatus of International Patent Publication No. WO 2015-072233, a configuration is disclosed that includes a tube body that has a corrugated tube and is extended in a long axis direction, a drive source disposed on the proximal side of this tube body, a driven member disposed on the distal side of the tube body, and a transmitting member that is provided in the tube body along the long axis of the tube body and is rotated around the long axis by a driving force of an electric motor or the like that is the drive source to transmit the rotation to the driven member.

Regarding the conventional insertion apparatus disclosed in International Patent Publication No. WO 2015-072233, a technique is disclosed that is for preventing a situation in which a rotational drive source included in the electric mechanism part or the driven member is disposed earlier than the transmitting member that transmits a rotational force of the rotational drive source to the driven member and the tube body having the corrugated tube is broken due to a twisting force from the rotational drive source or a twisting force from the driven member, without impairing functions possessed by the electric mechanism part.

Incidentally, with the conventional insertion apparatus like that in International Patent Publication No. WO 2015-072233, the insertion portion bends into various shapes according to the flexion state of a body cavity, the movability, and so forth when the insertion portion is inserted into the body cavity. For this reason, in the conventional insertion apparatus, resistance according to the bending shape of the driven member that rotates is added and the rotation of the driven member stops in some cases when the driving force by the drive source is small.

Furthermore, in the conventional insertion apparatus, increase in the size of the drive source becomes necessary when increasing the output power of rotational torque generated by the drive source is attempted to prevent the stop of the rotation of the driven member.

However, in the conventional insertion apparatus, the drive source such as an electric motor is provided in the operation unit. Thus, there is a problem that the size and weight of the operation unit increase when the larger drive source is provided.

In the case of increasing the rotational torque of the drive source by a reducer or the like, a problem has arisen such that the size of the operation unit is increased when the reducer is introduced on the drive source side and the diameter of the insertion portion increases when the reducer is introduced on the side of the driven member such as a rotating part.

### BRIEF SUMMARY OF EMBODIMENTS

The present disclosure provides an insertion apparatus comprising an insertion portion having a tubular body freely rotates around a longitudinal axis over an outer circumferential surface. The insertion portion is flexible and configured to be inserted into a body cavity. A drive source is configured to rotate the tubular body. A part of the insertion portion includes a predetermined flexural rigidity to which the tubular body being mounted thereto. The part of the insertion portion is formed of a structure that is configured in such a manner that bending of the tubular body is not caused beyond a predetermined bending angle based on the predetermined flexural rigidity so as to avoid stop of rotation of the tubular body by a driving force of the drive source even when an external force that intends to keep a bending shape of the body cavity is received from a wall of the body cavity in contact.

### BRIEF DESCRIPTION OF THE DRAWINGS

The technology disclosed herein, in accordance with one or more various embodiments, is described in detail with reference to the following figures. The drawings are provided for purposes of illustration only and merely depict typical or example embodiments of the disclosed technology. These drawings are provided to facilitate the reader's understanding of the disclosed technology and shall not be considered limiting of the breadth, scope, or applicability thereof. It should be noted that for clarity and ease of illustration these drawings are not necessarily made to scale.

FIG. 1 is a diagram depicting endoscope apparatus that is insertion apparatus according to one aspect of the present disclosure.

FIG. 2 is a diagram depicting a configuration that transmits a rotational driving force to a rotating unit according to the one aspect of the present disclosure.

FIG. 3 is a diagram depicting the configuration of a bending part, a first flexible tube part, a second flexible tube part, and the rotating unit according to the one aspect of the present disclosure.

FIG. 4 is a diagram depicting the configuration of the second flexible tube part, a third flexible tube part, a base part, and the rotating unit according to the one aspect of the present disclosure.

FIG. 5 is a sectional view along line V-V in FIG. 4 according to the one aspect of the present disclosure.

FIG. 6 is an exploded perspective view in which the first flexible tube part and the second flexible tube part are disassembled on each member basis according to the one aspect of the present disclosure.

FIG. 7 is an exploded perspective view that depicts a first form of a spiral tube and in which a tube part is disassembled on each member basis according to the one aspect of the present disclosure.

FIG. 8 is a side view depicting the rotating unit according to the one aspect of the present disclosure.

FIG. 9 is a sectional view of the tube part according to the one aspect of the present disclosure.

FIG. 10 is a side view depicting the state in which an insertion portion including the rotating unit is bent according to the one aspect of the present disclosure.

FIG. 11 is a sectional view of a bent corrugated tube according to the one aspect of the present disclosure.

FIG. 12 is a side view that depicts a second form of the spiral tube and depicts the rotating unit according to the one aspect of the present disclosure.

FIG. 13 is a sectional view of the tube part according to the one aspect of the present disclosure.

FIG. 14 is a side view depicting the state in which the insertion portion including the rotating unit is bent according to the one aspect of the present disclosure.

FIG. 15 is a sectional view of a bent helical tube according to the one aspect of the present disclosure.

FIG. 16 is a side view that depicts a third form of the spiral tube and depicts the rotating unit according to the one aspect of the present disclosure.

FIG. 17 is a sectional view of the tube part according to the one aspect of the present disclosure.

FIG. 18 is a side view depicting the state in which the insertion portion including the rotating unit is bent according to the one aspect of the present disclosure.

FIG. 19 is a side view that depicts a first form of the second flexible tube part and depicts the second flexible tube part to which the rotating unit is mounted according to the one aspect of the present disclosure.

FIG. 20 is a sectional view of the second flexible tube part according to the one aspect of the present disclosure.

FIG. 21 is a side view depicting the state in which the insertion portion including the rotating unit is bent according to the one aspect of the present disclosure.

FIG. 22 is a sectional view of a bent helical tube according to the one aspect of the present disclosure.

FIG. 23 is a side view depicting the second flexible tube part of the second form to which the rotating unit of the spiral tube is mounted according to the one aspect of the present disclosure.

FIG. 24 is a sectional view of the second flexible tube part according to the one aspect of the present disclosure.

FIG. 25 is a side view depicting the state in which the second flexible tube part to which the rotating unit is mounted is bent according to the one aspect of the present disclosure.

FIG. 26 is a sectional view of a bent corrugated tube according to the one aspect of the present disclosure.

FIG. 27 is a side view that depicts a third form of the second flexible tube part and depicts the second flexible tube part to which the rotating unit is mounted according to the one aspect of the present disclosure.

FIG. 28 is a sectional view of the second flexible tube part according to the one aspect of the present disclosure.

FIG. 29 is a side view depicting the state in which the second flexible tube part including the rotating unit is bent according to the one aspect of the present disclosure.

### DETAILED DESCRIPTION OF THE EMBODIMENTS

In the following description, various embodiments of the technology will be described. For purposes of explanation, specific configurations and details are set forth in order to provide a thorough understanding of the embodiments. However, it will also be apparent to one skilled in the art that the technology disclosed herein may be practiced without the specific details. Furthermore, well-known features may be omitted or simplified in order not to obscure the embodiment being described.

An embodiment of the present disclosure will be described below with reference to the drawings.

In the respective diagrams used for the following description, constituent elements whose scale is made different on each constituent element basis also exist in order to cause each constituent element to have such a size as to be recognizable on the drawing. That is, the present disclosure is not limited to only the numbers of constituent elements, the shapes of constituent elements, the ratio of the sizes of constituent elements, and the relative positional relationship among the respective constituent elements described in these diagrams.

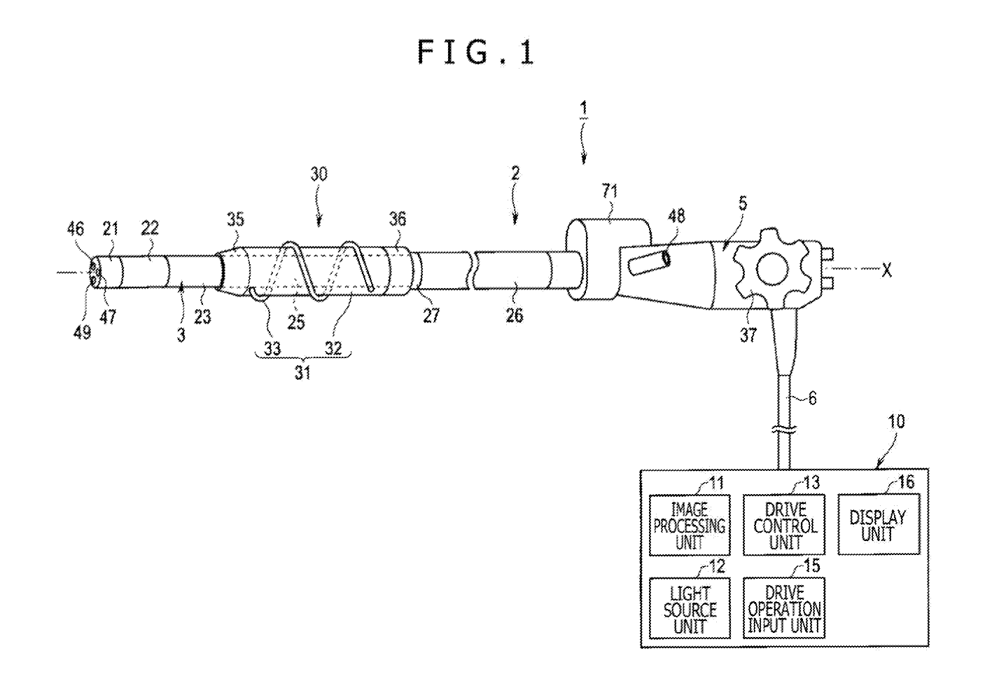

The embodiment of the present disclosure will be described with reference to FIG. 1 to FIG. 6.

An embodiment of endoscope apparatus that is insertion apparatus of one aspect of the present disclosure will be described with reference to the drawings.

As depicted in FIG. 1, endoscope apparatus 1 has a longitudinal axis X. Description will be made below in such a manner that the extension side of an insertion portion 3 as one side of a direction parallel to the longitudinal axis X of an endoscope 2 is defined as the distal direction and the side of an operation unit 5 in the opposite direction to the distal direction is defined as the proximal direction. Furthermore, the distal direction and the proximal direction are axis-parallel directions parallel to the longitudinal axis X.

The endoscope apparatus 1 includes the endoscope 2 that is insertion apparatus. The endoscope 2 includes the insertion portion, or endoscope insertion portion, 3 extended along the longitudinal axis X, the operation unit, or endoscope operation unit, 5 provided on the proximal direction side relative to the insertion portion 3, and a peripheral unit 10.

The peripheral unit 10 includes an image processing unit 11 such as an image processor, a light source unit 12 including a light source such as a lamp, a drive control unit 13 that is control apparatus including a power supply, a storing unit such as a memory, and a CPU or ASIC for example, an drive operation input unit 15 that is buttons, foot switches, and so forth, and a display unit 16 such as a monitor.

The insertion portion 3 of the endoscope 2 is extended along the longitudinal axis X and is inserted into a body cavity at the time of use of the endoscope apparatus 1. This insertion portion 3 includes a distal end forming part 21 that forms the distal end of the insertion portion 3, a bending part 22 provided on the proximal direction side relative to the distal end forming part 21, a first flexible tube part 23 provided on the proximal direction side relative to the bending part 22, a second flexible tube part 25 provided on the proximal direction side relative to the first flexible tube part 23, and a third flexible tube part 26 provided on the proximal direction side relative to the second flexible tube part 25.

A base part 27 is provided between the second flexible tube part 25 and the third flexible tube part 26 along the axis-parallel direction parallel to the longitudinal axis X. The second flexible tube part 25 is joined to the third flexible tube part 26 with the intermediary of the base part 27.

Here, in the section orthogonal to the longitudinal axis X, such a direction as to get further away from the longitudinal axis X is defined as the outer circumference direction, or axis-separated direction, and the central direction toward the longitudinal axis X is defined as the inner circumference direction, or axis-oriented direction.

In the insertion portion 3, a rotating unit 30 that has a tubular shape and is of a disposable, or expendable, type here is provided on the outer circumference direction side. That is, in the state in which the insertion portion 3 is inserted in the rotating unit 30, this rotating unit 30 is mounted to the second flexible tube part 25.

In the endoscope 2, in the state in which the rotating unit 30 is mounted to the insertion portion 3, the rotating unit 30 rotates around the longitudinal axis X relative to the insertion portion 3 by transmission of a rotational driving force thereto.

The rotating unit 30 includes a spiral tube 31 extended along the longitudinal axis X. The spiral tube 31 includes a tube part 32 and a fin part 33 extended on the outer circumferential surface of this tube part 32. The configuration of this tube part 32 will be described in detail later. In the spiral tube 31, the tube part 32 itself may by a corrugated tube.

The fin part 33 is extended from the proximal direction side to the distal direction side with a helical shape, with the longitudinal axis X being the center. A distal-side tubular part 35 is provided on the distal direction side of the spiral tube 31 in the rotating unit 30.

This distal-side tubular part 35 is formed into a tapered shape in which the outer diameter becomes smaller as the position gets closer to the distal direction side. Furthermore, a proximal-side tubular part 36 with a tubular shape is provided on the proximal direction side of the spiral tube 31.

In the state in which the fin part 33 of the spiral tube 31 is pressed in the inner circumference direction by a body cavity wall or the like, the rotating unit 30 rotates around the longitudinal axis X. Thereby, a propulsive force in the distal direction or the proximal direction acts on the insertion portion 3 and the rotating unit 30.

Specifically, the movability in the insertion direction of the insertion portion 3, or distal direction, in a body cavity such as inside of a small intestine or inside of a large intestine is improved due to the propulsive force in the distal direction, and the movability in the withdrawal direction of the insertion portion 3, or proximal direction, in the body cavity is improved due to the propulsive force in the proximal direction.

One end of a universal cord 6 is connected to the operation unit 5 of the endoscope 2. The other end of the universal cord 6 is connected to the peripheral unit 10. On the outer surface of the operation unit 5, a bending operation knob 37 to which bending operation of the bending part 22 is input is provided.

Furthermore, a procedure instrument insertion part 48 into which a procedure instrument such as forceps is inserted is provided on the outer surface of the operation unit 5. This procedure instrument insertion part 48 communicates with a channel tube 43 (see FIG. 3) disposed in the insertion portion 3.

Specifically, the channel tube 43 passes through the inside of the insertion portion 3 and the inside of the operation unit 5 and one end thereof is connected to the procedure instrument insertion part 48. Furthermore, the procedure instrument inserted from the procedure instrument insertion part 48 passes through the inside of the channel tube 43 and protrudes in the distal direction from an opening 49 of the distal end forming part 21. Then, procedure by the procedure instrument is carried out in the state in which the procedure instrument protrudes from the opening 49 of the distal end forming part 21.

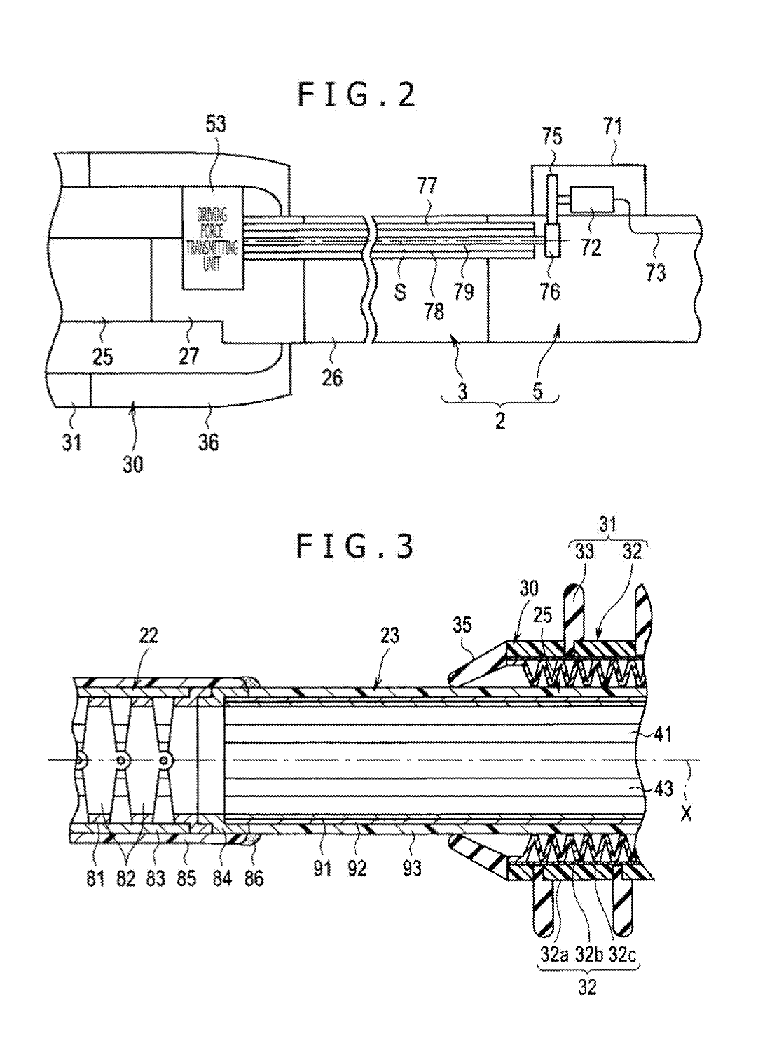

A motor housing 71 is joined to the operation unit 5. An electric motor 72 (see FIG. 2) that is a drive source is housed inside the motor housing 71.

As depicted in FIG. 2, one end of a motor cable 73 is connected to the electric motor 72 housed in the motor housing 71 provided on the operation unit 5. The motor cable 73 is extended to pass through the inside of the operation unit 5 and the inside of the universal cord 6 and the other end thereof is connected to the drive control unit 13 of the peripheral unit 10.

The electric motor 72 is driven by being supplied with power from the drive control unit 13 through the motor cable 73. Furthermore, due to the driving of the electric motor 72, a rotational driving force that rotates the rotating unit 30 is generated. A relay gear 75 is attached to the electric motor 72. Moreover, a drive gear 76 that meshes with the relay gear 75 is provided inside the operation unit 5.

As depicted in FIG. 3, inside the insertion portion 3, an imaging cable 41, a light guide 42, and the above-described channel tube 43 are extended along the longitudinal axis X.

Furthermore, the bending part 22 of the insertion portion 3 includes a bending tube 81. This bending tube 81 includes plural bending pieces 82 made of a metal.

Each bending piece 82 is pivotally joined to the adjacent bending piece 82. In the bending part 22, the outer circumference direction side of the bending tube 81 is covered by a bending reticular tube 83 that is a bending blade. In the bending reticular tube 83, wires (not depicted) made of a metal are woven into a mesh shape.

Moreover, in the bending part 22, the outer circumference direction side of the bending reticular tube 83 is covered by a bending envelope 85. The bending envelope 85 is formed of fluorine rubber for example.

An imaging element (not depicted) that images a subject is provided inside the distal end forming part 21, or distal part, of the insertion portion 3. This imaging element carries out imaging of a subject through an observation window 46 that is depicted in FIG. 1 and is provided at the distal end forming part 21 of the endoscope 2.

One end of the imaging cable 41 is connected to the imaging element. The imaging cable 41 is extended to pass through the inside of the insertion portion 3, the inside of the operation unit 5, and the inside of the universal cord 6 and the other end thereof is connected to the image processing unit 11 of the peripheral unit 10 depicted in FIG. 1.

Image processing of a subject image obtained by the imaging is executed by the image processing unit 11, so that an image of the subject is generated. Then, the generated image of the subject is displayed on the display unit 16 (see FIG. 1).

Furthermore, the light guide 42 is extended to pass through the inside of the insertion portion 3, the inside of the operation unit 5, and the inside of the universal cord 6 and is connected to the light source unit 12 of the peripheral unit 10. Light emitted from the light source unit 12 is guided by the light guide 42 and a subject is irradiated with the light from an illumination window 47 at the distal part, or distal end forming part 21, of the insertion portion 3 depicted in FIG. 1.

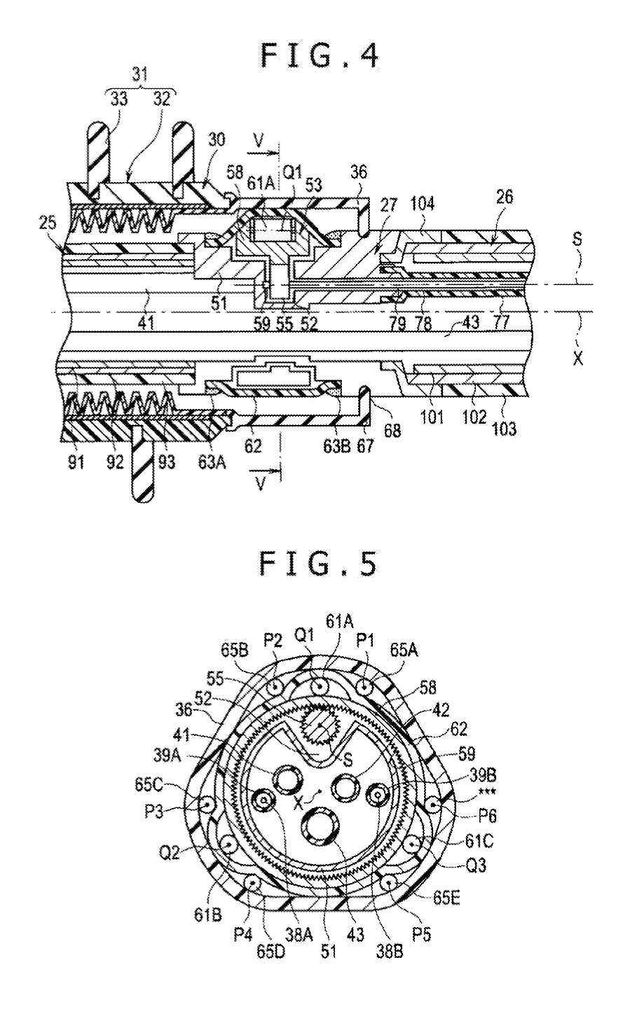

As depicted in FIG. 4, at the base part 27, a support member 51 formed from a metal is provided. The proximal part of the second flexible tube part 25 is joined to the distal part of the support member 51.

Furthermore, the distal part of the third flexible tube part 26 is joined to the proximal part of the support member 51. Due to this, the second flexible tube part 25 and the third flexible tube part 26 are connected via the base part 27.

As depicted in FIG. 4 and FIG. 5, a hollow part 52 is defined by the support member 51 in the base part 27. Furthermore, a driving force transmitting unit 53 is attached to the support member 51.

The driving force transmitting unit 53 is disposed in the hollow part 52. Furthermore, a rotational driving force that rotates the rotating unit 30 is transmitted to the driving force transmitting unit 53 and thereby the driving force transmitting unit 53 is driven. The driving force transmitting unit 53 includes a drive gear 55.

Moreover, the driving force transmitting unit 53 includes a rotating tubular member 58. This rotating tubular member 58 is attached to the base part 27 in the state in which the support member 51 is inserted in the rotating tubular member 58. The rotating tubular member 58 can freely rotate around the longitudinal axis X relative to the insertion portion 3, or base part 27.

Here, the two directions in which the rotating unit 30 rotates are defined as the direction around the longitudinal axis X. On the inner circumferential surface of the rotating tubular member 58, an inner circumferential gear part 59 is provided across the whole circumference regarding the direction around the longitudinal axis X. The inner circumferential gear part 59 meshes with the drive gear 55.

In the present embodiment, three inside rollers 61A to 61C are attached to the rotating tubular member 58. The inside rollers 61A to 61C are each disposed separately from each other by a predetermined interval in the direction around the longitudinal axis X.

The respective inside rollers 61A to 61C have corresponding roller axes Q1 to Q3. The respective inside rollers 61A to 61C can freely rotate relative to the rotating tubular member 58, with the corresponding roller axes Q1 to Q3 being the center.

Furthermore, the inside rollers 61A to 61C can each freely rotate integrally with the rotating tubular member 58 around the longitudinal axis relative to the insertion portion 3, or base part 27.

The outer circumference direction side of the rotating tubular member 58 and the inside rollers 61A to 61C is covered by a cover member 62 with a tubular shape. The distal end of the cover member 62 is fixed to the outer circumferential surface of the support member 51 with the intermediary of a bonding part 63A such as an adhesive and the proximal end of the cover member 62 is fixed to the outer circumferential surface of the support member 51 with the intermediary of a bonding part 63B such as an adhesive.

By the cover member 62, the hollow part 52 in which the driving force transmitting unit 53 is disposed is separated from the outside of the insertion portion 3. At the fixing position of the distal end of the cover member 62 and the fixing position of the proximal end of the cover member 62, watertightness between the support member 51 and the cover member 62 is kept.

Due to this, the flow of a liquid from the outside of the insertion portion 3 into the hollow part 52 and the driving force transmitting unit 53 is prevented. Furthermore, at the sites at which the inside rollers 61A to 61C are located, the cover member 62 protrudes in the outer circumference direction in the direction around the longitudinal axis X.

The cover member 62 is fixed to the insertion portion 3 and the rotating tubular member 58 and the inside rollers 61A to 61C can each freely rotate around the longitudinal axis X relative to the cover member 62.

As depicted in FIG. 5, six outside rollers 65A to 65F are attached to the inner circumferential surface of the proximal-side tubular part 36. The outside rollers 65A to 65F are located on the outer circumference direction side of the cover member 62.

In the state in which the rotating unit 30 is mounted to the insertion portion 3, in the direction around the longitudinal axis X, the inside roller 61A is located between the outside roller 65A and the outside roller 65B and the inside roller 61B is located between the outside roller 65C and the outside roller 65D.

Moreover, in the direction around the longitudinal axis X, the inside roller 61C is located between the outside roller 65E and the outside roller 65F. The respective outside rollers 65A to 65F have corresponding roller axes P1 to P6.

The respective outside rollers 65A to 65F can freely rotate relative to the cover member 62 and the proximal-side tubular part 36, with the corresponding roller axes P1 to P6 being the center. Furthermore, the outside rollers 65A to 65F can freely rotate integrally with the rotating unit 30 around the longitudinal axis X relative to the insertion portion 3, or base part 27.

Due to this configuration, the rotating tubular member 58 rotates around the longitudinal axis X when the driving force transmitting unit 53 is driven by a rotational driving force. This causes the inside roller 61A to press the outside roller 65A or the outside roller 65B.

Similarly, the inside roller 61B presses the outside roller 65C or the outside roller 65D and the inside roller 61C presses the outside roller 65E or the outside roller 65F.

Due to this, the driving force is transmitted from the inside rollers 61A to 61C to the outside rollers 65A to 65F of the rotating unit 30 and the rotating unit 30 rotates around the longitudinal axis X relative to the insertion portion 3 and the cover member 62.

As described above, the outside rollers 65A to 65F attached to the proximal-side tubular part 36 form a driving force receiving part that receives the rotational driving force from the driving force transmitting unit 53 that is driven.

The outside rollers 65A to 65F, which are this driving force receiving part, are provided on the proximal direction side relative to the spiral tube 31. Furthermore, in the state in which the rotating unit 30 is mounted to the insertion portion 3, the outside rollers 65A to 65F are located on the outer circumference direction side of the base part 27.

Because the respective inside rollers 61A to 61C rotate, with the corresponding roller axes Q1 to Q3 being the center, the friction between the respective inside rollers 61A to 61C and the cover member 62 becomes small.

Similarly, because the respective outside rollers 65A to 65F rotate, with the corresponding roller axes P1 to P6 being the center, the friction between the respective outside rollers 65A to 65F and the cover member 62 becomes small.

Thus, the rotational driving force is properly transmitted from the inside rollers 61A to 61C to the rotating unit 30 and the rotating unit 30 properly rotates.

In the proximal-side tubular part 36, a locking claw 67 that protrudes in the inner circumference direction is provided. Furthermore, in the support member 51 of the base part 27, a locking groove 68 is made across the whole circumference regarding the direction around the longitudinal axis.

The locking claw 67 is locked to the locking groove 68 and thereby movement of the rotating unit 30 along the longitudinal axis X relative to the insertion portion 3 is regulated. However, in the state in which the locking claw 67 is locked to the locking groove 68, the locking claw 67 can freely move in the direction around the longitudinal axis relative to the locking groove 68.

As depicted in FIG. 2 and FIG. 4, a guide tube 77 is extended along the longitudinal axis X inside the third flexible tube part 26 of the insertion portion 3. The distal end of the guide tube 77 is connected to the support member 51 of the base part 27.

A guide channel 78 is formed inside the guide tube 77. The distal end of the guide channel 78 communicates with the hollow part 52. In the guide channel 78, a drive shaft 79 that is a linear part is extended along a shaft axis S.

The rotational driving force generated by the electric motor 72 is transmitted to the drive shaft 79 via the relay gear 75 and the drive gear 76. Due to the transmission of the rotational driving force to the drive shaft 79, the drive shaft 79 rotates around the shaft axis S.

The distal end of the drive shaft 79 is connected to the drive gear 55 of the driving force transmitting unit 53. Due to the rotation of the drive shaft 79, the rotational driving force is transmitted to the driving force transmitting unit 53 and the driving force transmitting unit 53 is driven. Then, the rotational driving force is transmitted to the rotating tubular member 58 and thereby the rotational driving force is transmitted to the rotating unit 30 as described above. This rotates the rotating unit 30.

As depicted in FIG. 5, bending wires 38A and 38B are extended along the longitudinal axis X inside the insertion portion 3. The proximal ends of the bending wires 38A and 38B are connected to a pulley (not depicted) joined to the bending operation knob 37 inside the operation unit 5.

The distal ends of the bending wires 38A and 38B are connected to the distal part of the bending part 22. By bending operation with the bending operation knob 37, the bending wire 38A or the bending wire 38B is pulled and the bending part 22 bends. In the present embodiment, the bending part 22 is formed of only an active bending part that bends by bending operation.

The respective bending wires 38A and 38B are inserted in corresponding coils 39A and 39B. The proximal ends of the coils 39A and 39B are extended to the inside of the operation unit 5. Furthermore, the distal ends of the coils 39A and 39B are connected to the inner circumferential surface of the distal part of the first flexible tube part 23. In the present embodiment, the two bending wires 38A and 38B are provided and the bending part 22 can bend in two directions. However, for example four bending wires may be provided and the bending part 22 may be capable of bending in four directions.

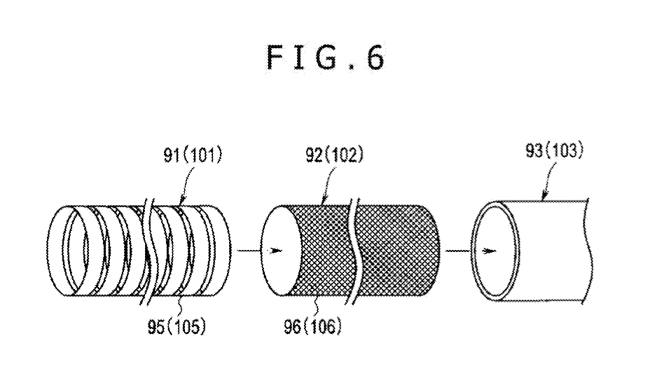

In the endoscope 2 of the present embodiment, as depicted in FIG. 6, the first flexible tube part 23 and the second flexible tube part 25 are formed of a first helical tube 91 that is a first flex tube, a first flexible reticular tube 92 that is a first flexible blade tube, and a first flexible envelope 93 that is an envelope tube.

The first helical tube 91, the first flexible reticular tube 92, and the first flexible envelope 93 are extended along the longitudinal axis X from the distal end of the first flexible tube part 23 to the proximal end of the second flexible tube part 25.

The outer circumference direction side of the first helical tube 91 is covered by the first flexible reticular tube 92 and the outer circumference direction side of the first flexible reticular tube 92 is covered by the first flexible envelope 93.

The first helical tube 91 includes a strip-shaped member 95 made of a metal. In the first helical tube 91, the strip-shaped member 95 is extended into a helical shape around the longitudinal axis X.

The first flexible reticular tube 92 includes wires 96 made of a metal. The wires 96 are woven in the first flexible reticular tube 92. The first flexible envelope 93 is formed of a resin material.

The proximal part of the bending tube 81 is fitted to a connecting tube 84 with a tubular shape (see FIG. 3) and the first helical tube 91 and the first flexible reticular tube 92 are fitted to the connecting tube 84 in the state of being inserted on the inner circumference direction side of the connecting tube 84.

Furthermore, the first flexible envelope 93 is bonded to the bending envelope 85 with the intermediary of a bonding part 86 such as an adhesive. The first flexible tube part 23 is joined to the bending part 22 in the above-described manner. As depicted in FIG. 4, the first helical tube 91, the first flexible reticular tube 92, and the first flexible envelope 93 are fitted to the support member 51 in the state of being inserted on the inner circumference direction side of the support member 51.

Thereby, the second flexible tube part 25 is joined to the base part 27. Furthermore, in the present embodiment, the first helical tube 91, the first flexible reticular tube 92, and the first flexible envelope 93 are extended in a continuous state between the first flexible tube part 23 and the second flexible tube part 25.

The third flexible tube part 26 is formed of a second helical tube 101 that is a second flex tube, a second flexible reticular tube 102 that is a second flexible blade tube, and a second flexible envelope 103 (reference numerals in parentheses in FIG. 6).

The second helical tube 101, the second flexible reticular tube 102, and the second flexible envelope 103 are extended along the longitudinal axis X from the distal end of the third flexible tube part 26 to the proximal end of the third flexible tube part 26. The outer circumference direction side of the second helical tube 101 is covered by the second flexible reticular tube 102 and the outer circumference direction side of the second flexible reticular tube 102 is covered by the second flexible envelope 103.

The proximal end of the support member 51 is fitted to a connecting member 104. The second helical tube 101 and the second flexible reticular tube 102 are fitted to the connecting member 104 in the state of being inserted on the inner circumference direction side of the connecting member 104 (see FIG. 4). Due to this, the third flexible tube part 26 is joined to the base part 27.

In the second helical tube 101, a strip-shaped member 105 made of a metal is extended into a helical shape centered at the longitudinal axis X. Furthermore, in the second flexible reticular tube 102, wires 106 made of a metal are woven. The second flexible envelope 103 is formed of a resin material.

Here, various configurations of the spiral tube 31 will be described in detail below.

### First Form of Spiral Tube

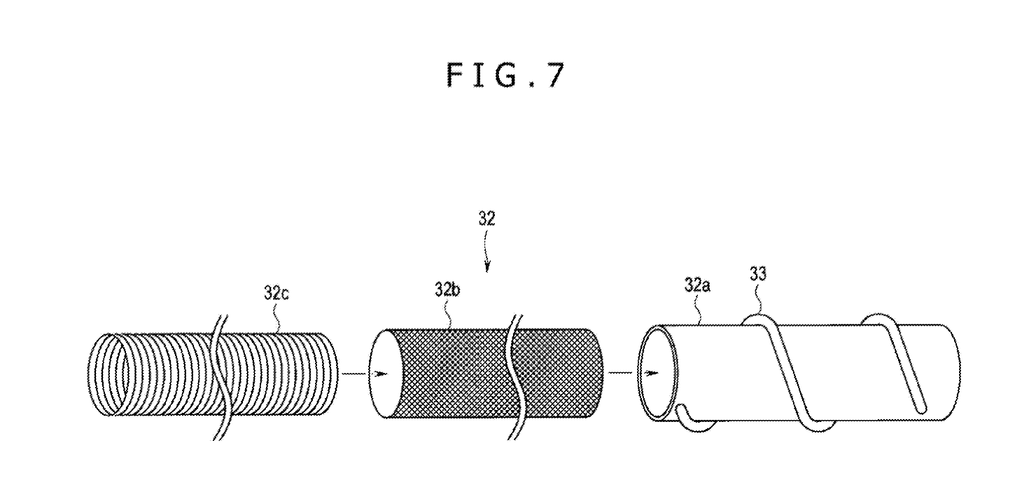

Based on FIG. 7 to FIG. 11, a first form of the configuration of the tube part 32 occupying a large part of the spiral tube 31 will be described below.

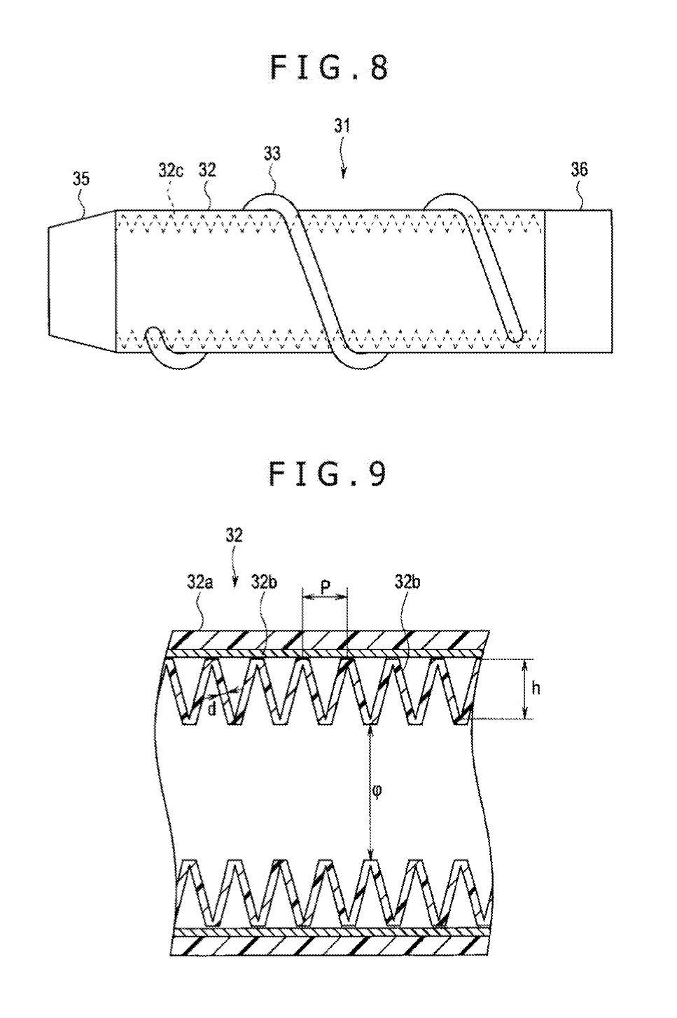

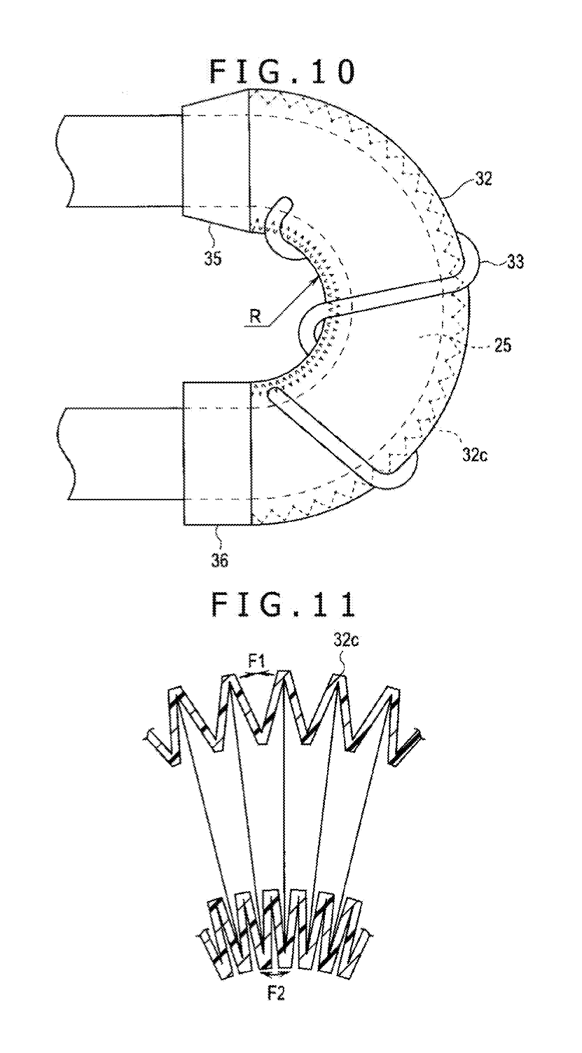

FIG. 7 is an exploded perspective view that depicts the first form of the spiral tube and in which the tube part is disassembled on each member basis. FIG. 8 is a side view depicting the rotating unit. FIG. 9 is a sectional view of the tube part. FIG. 10 is a side view depicting the state in which the insertion portion including the rotating unit is bent. FIG. 11 is a sectional view of a bent corrugated tube.

As depicted in FIG. 7 and FIG. 8, the tube part 32 occupying a large part of the spiral tube 31 of the present form includes a covering tube 32a serving as an outer layer, a flexible reticular tube 32b serving as a middle layer, and a corrugated tube 32c serving as an inner layer.

In the tube part 32, the outer circumferential side of the corrugated tube 32c is covered by the flexible reticular tube 32b and the outer circumferential side of this flexible reticular tube 32b is covered by the covering tube 32a on which the fin part 33 is provided.

The flexible reticular tube 32b is a metal mesh tube into which wires made of a metal are woven. An elastic tube may be used instead of the flexible reticular tube 32b in the tube part 32. Furthermore, the corrugated tube 32c is a so-called accordion tube.

Based on these covering tube 32a, flexible reticular tube 32b, and corrugated tube 32c, the flexural rigidity of the whole of the tube part 32 is configured.

Specifically, in the tube part 32 of the present form, predetermined flexural rigidity based on the corrugated tube 32c is configured in addition to predetermined flexural rigidity of the covering tube 32a and the flexible reticular tube 32b.

This flexural rigidity of the corrugated tube 32c is decided based on various parameters (constituent elements based on a structure of various kinds of members) such as pitch P between top parts, thickness d, height h of concavities and convexities, inner diameter φ, and materials as depicted in FIG. 9.

Here, in FIG. 10, the state in which the spiral tube 31 is bent at a predetermined bending angle R, here 180°, is depicted. In this case, in the corrugated tube 32c, as depicted in FIG. 11, a sum (F1+F2) of bending stress F1 based on a tensile force generated on the bending outer side on which the corrugated tube 32c intends to return to the straight line state based on the flexural rigidity and bending stress F2 based on a repulsive force generated on the bending inner side per one pitch P between top parts is generated.

Furthermore, in the whole of the corrugated tube 32c, stress of a product {nP×(F1+F2)} of the number of pitches P (n) and the bending stress (F1+F2) per one pitch P is generated and the flexural rigidity is decided.

As above, for the spiral tube 31, the predetermined flexural rigidity in the state in which the spiral tube 31 is bent at the predetermined bending angle R, here 180° for example, is configured based on the predetermined flexural rigidity of the covering tube 32a and the flexible reticular tube 32b and the predetermined flexural rigidity based on the corrugated tube 32c, and the predetermined flexural rigidity is decided based on the above-described various kinds of parameters (constituent elements based on a structure of various kinds of members).

The predetermined bending angle R is not limited to 180° and can be configured as appropriate to a predetermined angle with which the spiral tube 31 rotates without stopping with respect to the rotational torque, or drive torque, by the electric motor 72, which is the drive source.

### Second Form of Spiral Tube

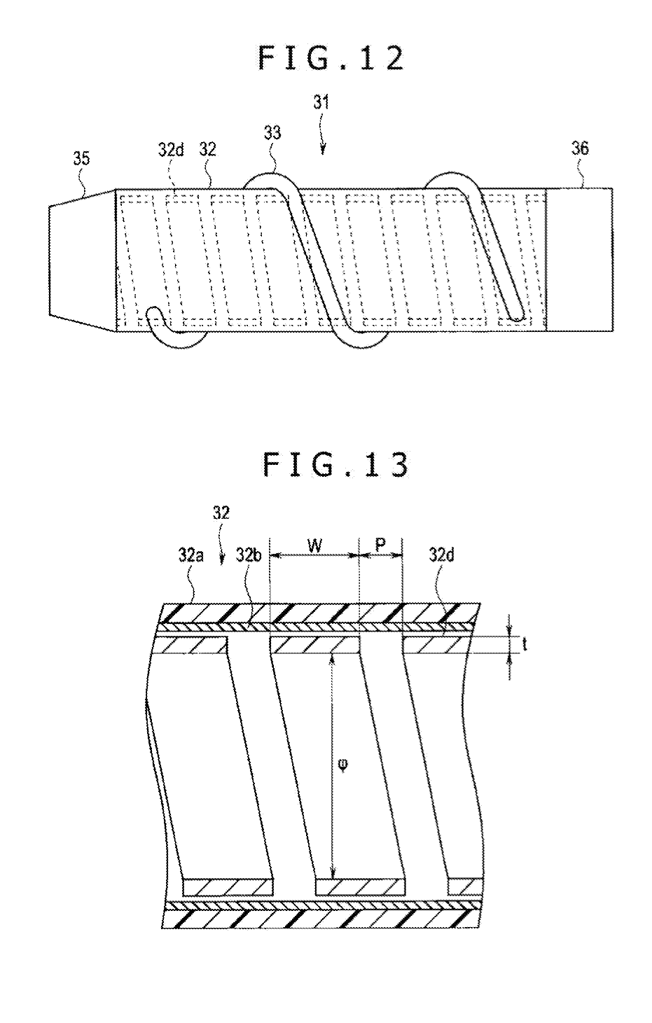

Based on FIG. 12 to FIG. 15, a second form of the configuration of the tube part 32 occupying a large part of the spiral tube 31 will be described below.

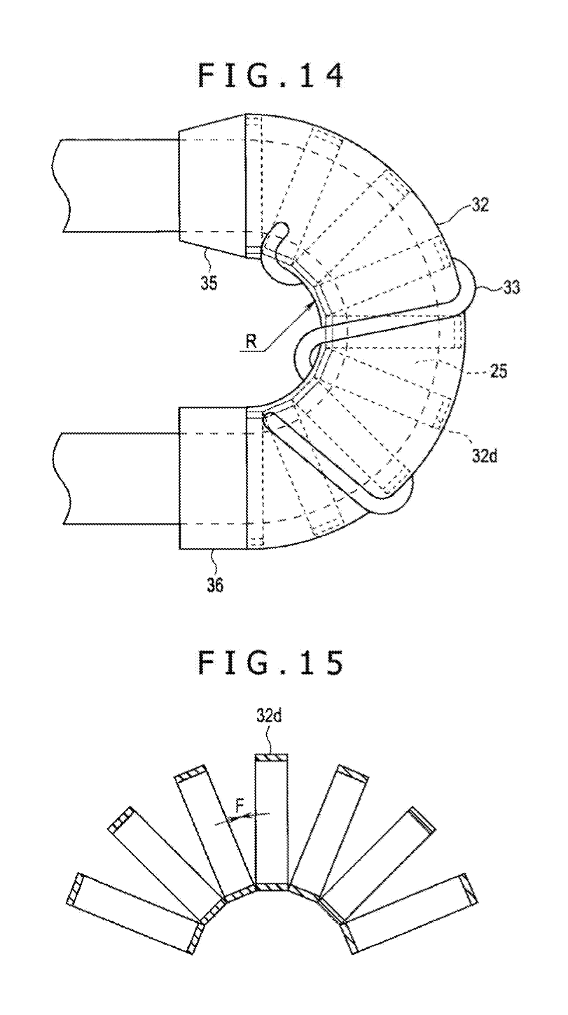

FIG. 12 is a side view that depicts the second form of the spiral tube and depicts the rotating unit. FIG. 13 is a sectional view of the tube part. FIG. 14 is a side view depicting the state in which the insertion portion including the rotating unit is bent. FIG. 15 is a sectional view of a bent helical tube.

As depicted in FIG. 12 and FIG. 13, the tube part 32 occupying a large part of the spiral tube 31 of the present form includes the covering tube 32a serving as an outer layer, the flexible reticular tube 32b serving as a middle layer, and here a helical tube 32d serving as an inner layer instead of the corrugated tube 32c.

In the tube part 32, the outer circumferential side of the helical tube 32d is covered by the flexible reticular tube 32b and the outer circumferential side of this flexible reticular tube 32b is covered by the covering tube 32a on which the fin part 33 is provided. The helical tube 32d is a tube body that is formed by winding a strip-shaped member made of a metal into a helical shape and has flexibility.

Based on these covering tube 32a, flexible reticular tube 32b, and helical tube 32d, the flexural rigidity of the whole of the tube part 32 is configured.

Specifically, in the tube part 32 of the present form, predetermined flexural rigidity based on the helical tube 32d is configured in addition to predetermined flexural rigidity of the covering tube 32a and the flexible reticular tube 32b.

This flexural rigidity of the helical tube 32d is decided based on various parameters (constituent elements based on a structure of various kinds of members) such as pitch P of the wound strip-shaped member, width W, thickness t, inner diameter φ, and materials as depicted in FIG. 13.

In FIG. 14, the state in which the spiral tube 31 is bent at the predetermined bending angle R, here 180° for example, is depicted. In this case, in the helical tube 32d, as depicted in FIG. 15, bending stress F based on a tensile force generated on the bending outer side on which the helical tube 32d intends to return to the straight line state based on the flexural rigidity per one pitch P of the wound strip-shaped member is generated.

Furthermore, in the whole of the helical tube 32d, stress of a product (nP×F) of the number of pitches P (n) and the bending stress (F) per one pitch P is generated and the flexural rigidity is decided.

As above, for the spiral tube 31, the predetermined flexural rigidity in the state in which the spiral tube 31 is bent at the predetermined bending angle R, here 180°, is configured based on the predetermined flexural rigidity of the covering tube 32a and the flexible reticular tube 32b and the predetermined flexural rigidity based on the helical tube 32d, and the predetermined flexural rigidity is decided based on the above-described various kinds of parameters (constituent elements based on a structure of various kinds of members).

The predetermined bending angle R is not limited to 180° and can be configured as appropriate to a predetermined angle with which the spiral tube 31 rotates without stopping with respect to the rotational torque, or drive torque, by the electric motor 72, which is the drive source.

### Third Form of Spiral Tube

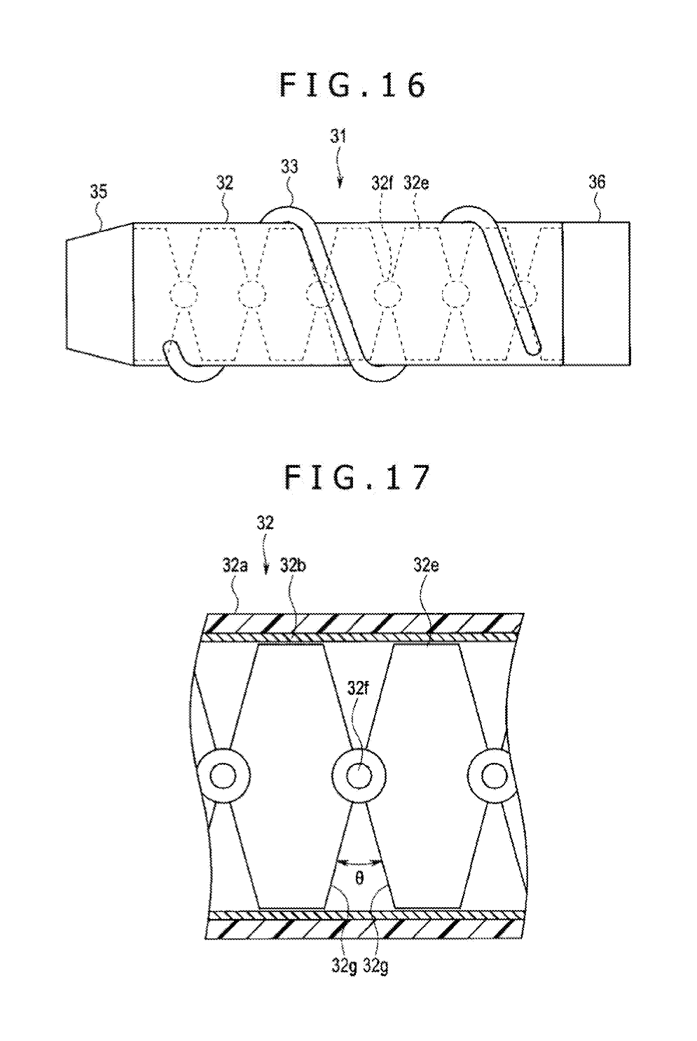

Based on FIG. 16 to FIG. 18, a third form of the configuration of the tube part 32 occupying a large part of the spiral tube 31 will be described below.

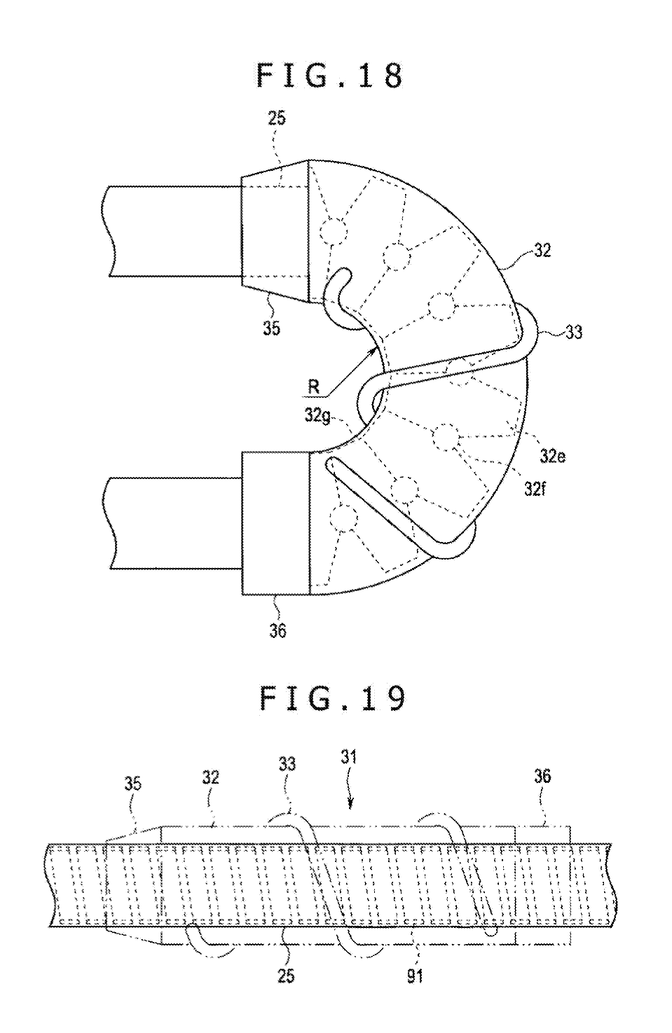

FIG. 16 is a side view that depicts the third form of the spiral tube and depicts the rotating unit. FIG. 17 is a sectional view of the tube part. FIG. 18 is a side view depicting the state in which the insertion portion including the rotating unit is bent.

As depicted in FIG. 16 and FIG. 17, the tube part 32 occupying a large part of the spiral tube 31 of the present form includes the covering tube 32a serving as an outer layer, the flexible reticular tube 32b serving as a middle layer, and here plural bending regulating pieces 32e serving as an inner layer instead of the corrugated tube 32c or the helical tube 32d.

In the tube part 32, the outer circumferential side of the plural bending regulating pieces 32e is covered by the flexible reticular tube 32b and the outer circumferential side of this flexible reticular tube 32b is covered by the covering tube 32a on which the fin part 33 is provided. The plural bending regulating pieces 32e are pivotally joined by pivotally-support parts 32f such as rivets and form a bending tube.

Here, the bending state of the whole of the tube part 32 is restricted by the plural bending regulating pieces 32e. The bending angle R thereof is defined through abutting of opposing end surfaces 32g of the plural bending regulating pieces 32e against each other and is determined by an angle θ made by two opposing end surfaces 32g in the straight line state.

In FIG. 18, the state in which the spiral tube 31 is bent at the predetermined bending angle R, here 180° for example, is depicted. In this case, the end surfaces 32g on the bending inner side in the plural bending regulating pieces 32e abut against each other and the maximum bending angle R is defined.

That is, the bending angle R of the spiral tube 31 is decided based on the shape of the plural bending regulating pieces 32e. For example, if two adjacent bending regulating pieces 32e are defined as one set, or one pair, the bending angle R of the spiral tube 31 is decided based on the product of the bending angle based on this one set of bending regulating pieces 32e and the number of pivotally-support parts 32f.

Although the configuration in which the bending tube obtained by joining the plural bending regulating pieces 32e bends in two directions is depicted in the diagram here, a configuration in which the position of the joint by the pivotally-support part 32f is changed in the circumferential direction so that the bending tube can three-dimensionally bend may be employed, of course.

The predetermined bending angle R is not limited to 180° and can be set as appropriate to a predetermined angle with which the spiral tube 31 rotates without stopping with respect to the rotational torque, or drive torque, by the electric motor 72, which is the drive source.

Next, description will be made in detail below about various configurations of the second flexible tube part 25 as the part of the insertion portion 3 to which the rotating unit 30 is mounted on the outer circumference direction side and to which the spiral tube 31 is mounted.

### First Form of Second Flexible Tube Part

Based on FIG. 19 to FIG. 22, a first form of the configuration of the second flexible tube part 25 will be described below.

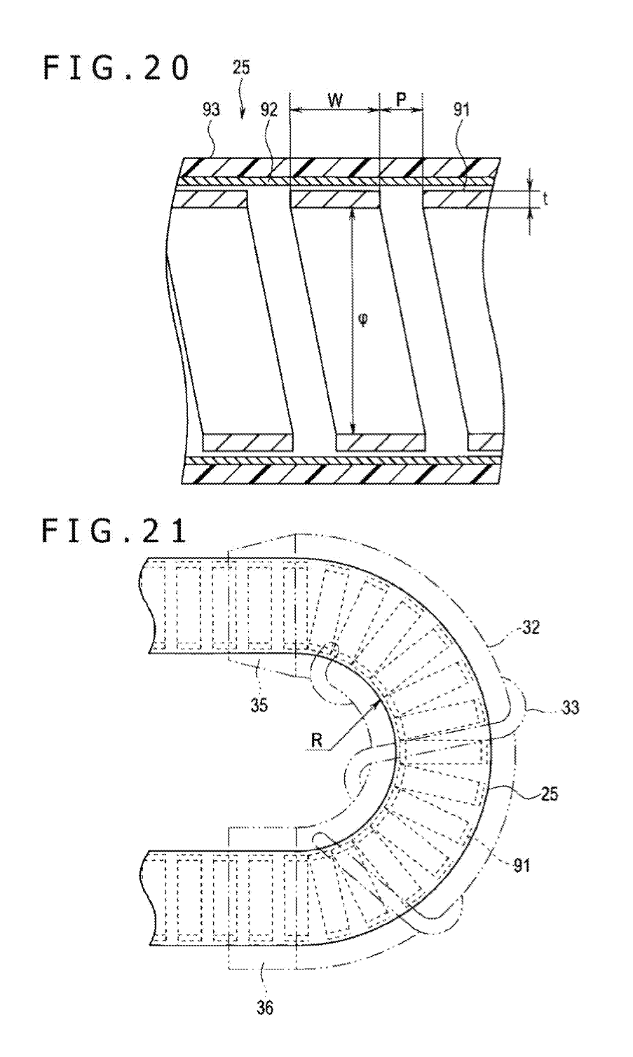

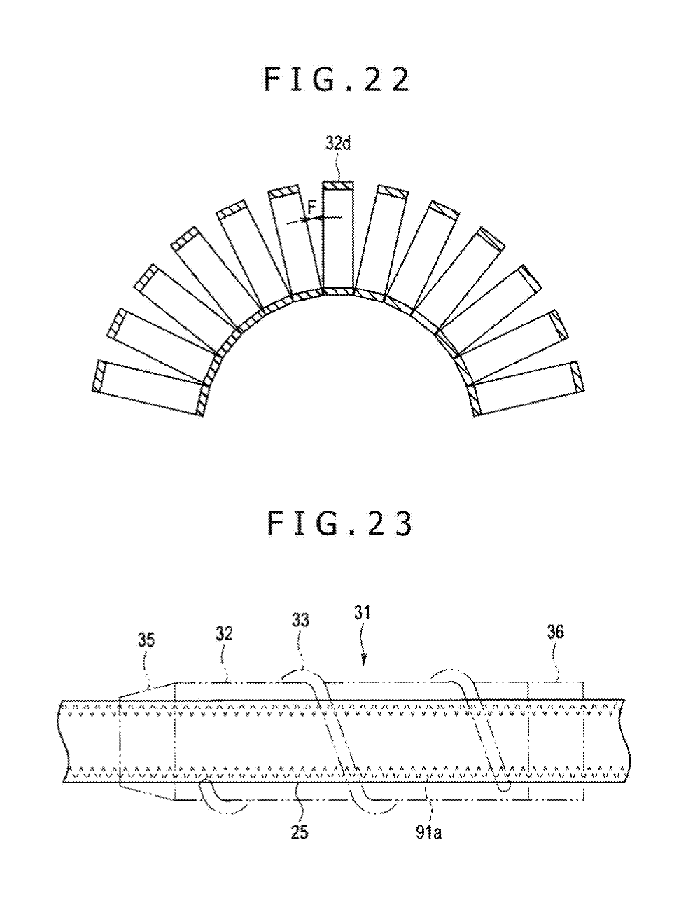

FIG. 19 is a side view that depicts the first form of the second flexible tube part and depicts the second flexible tube part to which the rotating unit is mounted. FIG. 20 is a sectional view of the second flexible tube part. FIG. 21 is a side view depicting the state in which the insertion portion including the rotating unit is bent. FIG. 22 is a sectional view of a bent helical tube.

As depicted in FIGS. 19 and 20 and as described above, the second flexible tube part 25 as the part of the insertion portion 3 to which the spiral tube 31 of the present form is mounted is configured to have the first helical tube 91 that is the first flex tube, the first flexible reticular tube 92 as a covering layer that is the first flexible blade tube, and the first flexible envelope 93 as a coat layer that is the envelope tube.

In the second flexible tube part 25, the outer circumferential side of the first helical tube 91 is covered by the first flexible reticular tube 92 and the outer circumferential side of this first flexible reticular tube 92 is covered by the first flexible envelope 93. An elastic tube may be used as the first flexible reticular tube 92.

The first helical tube 91 is a tube body that is formed by winding a strip-shaped member made of a metal into a helical shape and has flexibility. Based on these first helical tube 91, first flexible reticular tube 92, and first flexible envelope 93, the flexural rigidity of the whole of the second flexible tube part 25 is set.

Specifically, in the second flexible tube part 25 of the present form, predetermined flexural rigidity based on the first helical tube 91 is set in addition to predetermined flexural rigidity of the first flexible envelope 93 and the first flexible reticular tube 92 and the flexural rigidity of various kinds of built-in objects such as the imaging cable 41, the light guide 42, and the channel tube 43.

This flexural rigidity of the first helical tube 91 is decided based on various parameters (constituent elements based on a structure of various kinds of members) such as pitch P of the wound strip-shaped member, width W, thickness t, inner diameter φ, and materials as depicted in FIG. 20.

In FIG. 21, the state in which the first helical tube 91 to which the spiral tube 31 of the rotating unit 30 is mounted is bent at the predetermined bending angle R, here 180° for example, is depicted. In this case, in the first helical tube 91, as depicted in FIG. 22, bending stress F based on a tensile force generated on the bending outer side on which the first helical tube 91 intends to return to the straight line state based on the flexural rigidity per one pitch P of the wound strip-shaped member is generated.

Furthermore, in the whole of the first helical tube 91, stress of a product (nP×F) of the number of pitches P (n) and the bending stress (F) per one pitch P is generated and the flexural rigidity is decided.

As above, for the second flexible tube part 25, the predetermined flexural rigidity in the state in which the second flexible tube part 25 is bent at the predetermined bending angle R, here 180°, is set based on the predetermined flexural rigidity of the first flexible envelope 93 and the first flexible reticular tube 92 and the predetermined flexural rigidity based on the first helical tube 91, and the predetermined flexural rigidity is decided based on the above-described various kinds of parameters (constituent elements based on a structure of various kinds of members).

The predetermined bending angle R is not limited to 180° and can be set as appropriate to a predetermined angle with which the spiral tube 31 rotates without stopping with respect to the rotational torque, or drive torque, by the electric motor 72, which is the drive source.

### Second Form of Second Flexible Tube Part

Based on FIG. 23 to FIG. 26, the second form of the configuration of the second flexible tube part 25 will be described below.

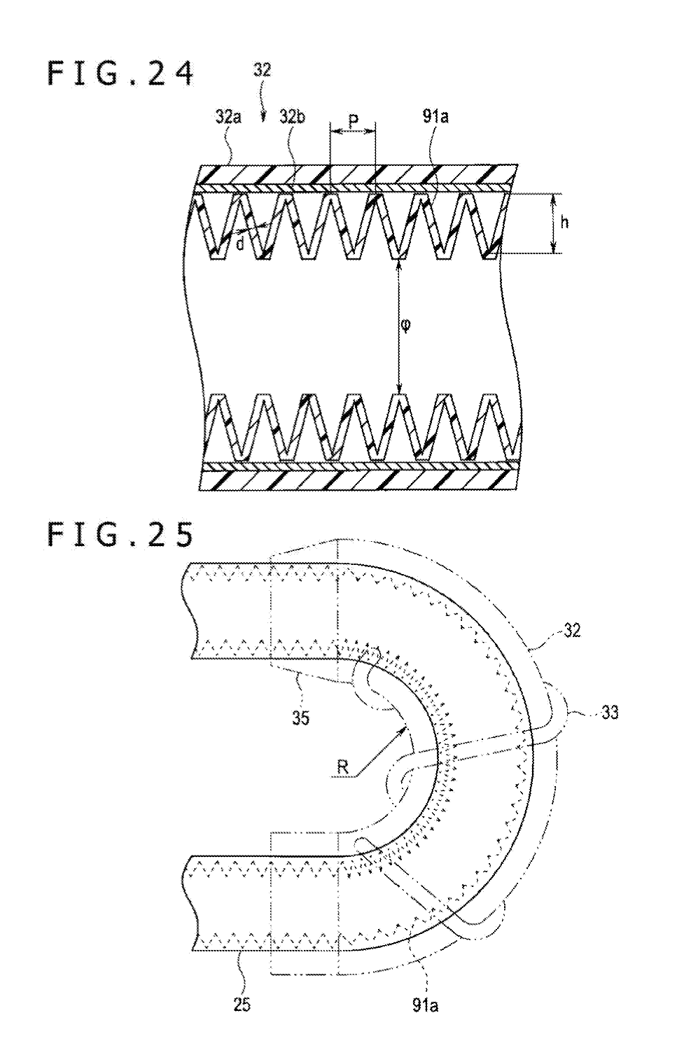

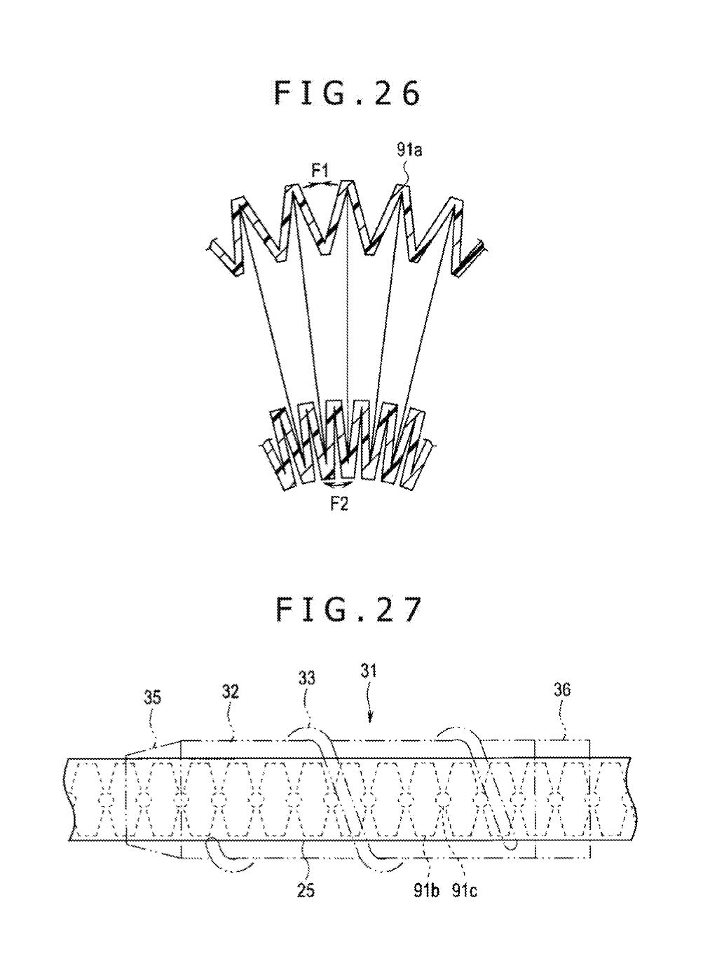

FIG. 23 is a side view depicting the second flexible tube part of the second form to which the rotating unit of the spiral tube is mounted. FIG. 24 is a sectional view of the second flexible tube part. FIG. 25 is a side view depicting the state in which the second flexible tube part to which the rotating unit is mounted is bent. FIG. 26 is a sectional view of a bent corrugated tube.

The second flexible tube part 25 of the present form as the part of the insertion portion 3 to which the spiral tube 31 is mounted is depicted in FIG. 23 and FIG. 24 and is configured to have a corrugated tube 91a instead of the first helical tube 91, the first flexible reticular tube 92 as the coating layer that is the first flexible blade tube, and the first flexible envelope 93 as the coat layer that is the envelope tube.

In the second flexible tube part 25, the outer circumferential side of the corrugated tube 91a is covered by the first flexible reticular tube 92 and the outer circumferential side of this first flexible reticular tube 92 is covered by the first flexible envelope 93. The first helical tube 91 is a tube body that is formed by winding a strip-shaped member made of a metal into a helical shape and has flexibility. An elastic tube may be used as the first flexible reticular tube 92. Furthermore, the corrugated tube 91a is a so-called accordion tube.

Based on these corrugated tube 91a, first flexible reticular tube 92, and first flexible envelope 93, the flexural rigidity of the whole of the second flexible tube part 25 is set.

Specifically, in the second flexible tube part 25 of the present form, predetermined flexural rigidity based on the corrugated tube 91a is set in addition to predetermined flexural rigidity of the first flexible reticular tube 92 and the first flexible envelope 93 and the flexural rigidity of various kinds of built-in objects such as the imaging cable 41, the light guide 42, and the channel tube 43.

This flexural rigidity of the corrugated tube 91a is decided based on various parameters (constituent elements based on a structure of various kinds of members) such as pitch P between top parts, thickness d, height h of concavities and convexities, inner diameter φ, and materials as depicted in FIG. 24.

Here, in FIG. 25, the state in which the second flexible tube part 25 is bent at the predetermined bending angle R, here 180°, is depicted. In this case, in the corrugated tube 91a, as depicted in FIG. 26, a sum (F1+F2) of bending stress F1 based on a tensile force generated on the bending outer side on which the corrugated tube 91a intends to return to the straight line state based on the flexural rigidity and bending stress F2 based on a repulsive force generated on the bending inner side per one pitch P between top parts is generated.

Furthermore, in the whole of the corrugated tube 91a, stress of a product {nP×(F1+F2)} of the number of pitches P (n) and the bending stress (F1+F2) per one pitch P is generated and the flexural rigidity is decided.

As above, for the second flexible tube part 25, the predetermined flexural rigidity in the state in which the second flexible tube part 25 is bent at the predetermined bending angle R, here 180° for example, is set based on the predetermined flexural rigidity of the first flexible reticular tube 92 and the first flexible envelope 93 and the predetermined flexural rigidity based on the corrugated tube 91a, and the predetermined flexural rigidity is decided based on the above-described various kinds of parameters (constituent elements based on a structure of various kinds of members).

The predetermined bending angle R is not limited to 180° and can be set as appropriate to a predetermined angle with which the spiral tube 31 rotates without stopping with respect to the rotational torque, or drive torque, by the electric motor 72, which is the drive source.

### Third Form of Second Flexible Tube Part

Based on FIG. 27 to FIG. 29, a third form of the configuration of the second flexible tube part 25 will be described below.

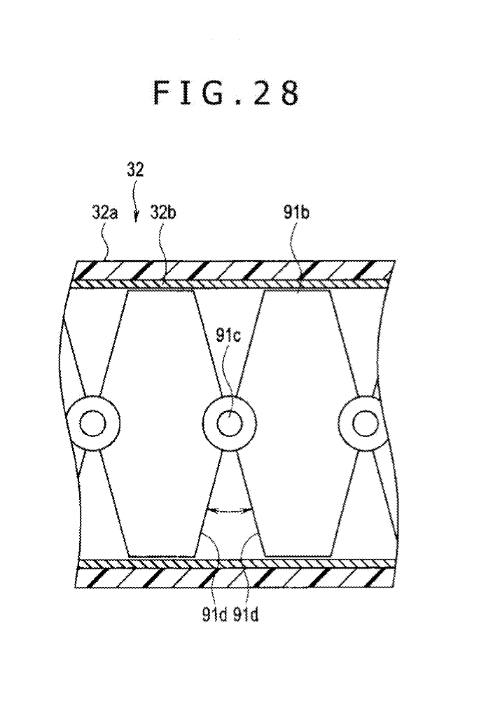

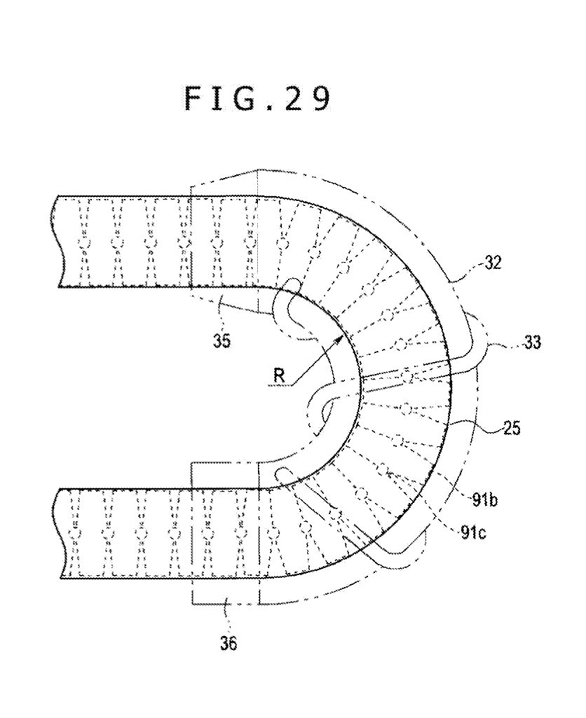

FIG. 27 is a side view that depicts the third form of the second flexible tube part and depicts the second flexible tube part to which the rotating unit is mounted. FIG. 28 is a sectional view of the second flexible tube part. FIG. 29 is a side view depicting the state in which the second flexible tube part including the rotating unit is bent.

As depicted in FIGS. 27 and 28, the second flexible tube part 25 as the part of the insertion portion 3 to which the spiral tube 31 of the present form is mounted is configured to have plural bending regulating pieces 91b forming a bending tube instead of the first helical tube 91 or the corrugated tube 91a, the first flexible reticular tube 92 as the covering layer that is the first flexible blade tube, and the first flexible envelope 93 as the coat layer that is the envelope tube.

In the second flexible tube part 25, the outer circumferential side of the plural bending regulating pieces 91b is covered by the first flexible reticular tube 92 and the outer circumferential side of this first flexible reticular tube 92 is covered by the first flexible envelope 93. The first helical tube 91 is a tube body that is formed by winding a strip-shaped member made of a metal into a helical shape and has flexibility. An elastic tube may be used as the first flexible reticular tube 92.

The plural bending regulating pieces 91b are pivotally joined by pivotally-support parts 91c such as rivets and form the bending tube.

Here, the bending state of the whole of the second flexible tube part 25 is restricted by the plural bending regulating pieces 91b. The bending angle R thereof is defined through abutting of opposing end surfaces 91d of the plural bending regulating pieces 91b against each other and is determined by an angle θ made by two opposing end surfaces 91d in the straight line state.

In FIG. 29, the state in which the second flexible tube part 25 is bent at the predetermined bending angle R, here 180° for example, is depicted. In this case, the end surfaces 91d on the bending inner side in the plural bending regulating pieces 91b abut against each other and the maximum bending angle R is defined.

That is, the bending angle R of the second flexible tube part 25 is decided based on the shape of the plural bending regulating pieces 91b. For example, if two adjacent bending regulating pieces 91b are defined as one set, or one pair, the bending angle R of the second flexible tube part 25 is decided based on the product of the bending angle based on this one set of bending regulating pieces 91b and the number of pivotally-support parts 91c.

Although the configuration in which the bending tube obtained by joining the plural bending regulating pieces 91b bends in two directions is depicted in the diagram here, a configuration in which the position of the joint by the pivotally-support part 91c is changed in the circumferential direction so that the bending tube can three-dimensionally bend may be employed, of course.

Regarding the endoscope apparatus 1 of the present embodiment configured as described above, description will be made about operation and effects of the endoscope apparatus 1 that is insertion apparatus including the rotating unit 30 and the endoscope 2 that is insertion apparatus.

When the endoscope apparatus 1 is used, the insertion portion 3 and the rotating unit 30 are inserted into a body cavity in the state in which the rotating unit 30 is mounted to the insertion portion 3. Then, by driving the electric motor 72 in the state in which the fin part 33 of the spiral tube 31 abuts against a body cavity wall, a rotational driving force is transmitted to the driving force transmitting unit 53 attached to the base part 27 of the insertion portion 3.

Then, the driving force transmitting unit 53 is driven and the outside rollers 65A to 65F, which are the driving force receiving part, receive the rotational driving force from the driving force transmitting unit 53. Thereby, the rotating unit 30 rotates around the longitudinal axis X.

Due to the rotation of the rotating unit 30 around the longitudinal axis X in the state in which the fin part 33 of the spiral tube 31 is pressed in the inner circumference direction by the body cavity wall and so forth, a propulsive force that causes the insertion portion 3 to advance in the distal direction or retreat in the proximal direction acts on the insertion portion 3 and the rotating unit 30.

At this time, in the endoscope apparatus 1 of the present embodiment, when the insertion portion 3 passes through a bending part of the body cavity, e.g. the pharyngeal region of the esophagus, which is an upper-side body cavity, from the oral cavity, the ileocecal valve existing near the cecum in the small intestine, the splenic flexure or hepatic flexure of the large intestine, which is a lower-side body cavity, from the anus, or the like, the spiral tube 31 of the rotating unit 30 does not excessively bend and stop of the rotation is prevented.

To state it differently, in the drive torque to drive the rotating unit 30 by the electric motor 72, transmission loss of various kinds of drive systems, such as loss of friction due to gear parts such as the drive gears 55 and 76 and the relay gear 75, loss of friction between the drive shaft 79 and the guide channel 78 and so forth, and loss of friction of the inside rollers 61A to 61C, the outside rollers 65A to 65F, and so forth against the proximal-side tubular part 36 or the cover member 62, are generated.

In addition to this transmission loss of drive systems, rotation loss such as frictional resistance due to bending of the spiral tube 31 is generated. For this reason, by keeping the total loss of the transmission loss of drive systems and the rotation loss due to bending of the spiral tube 31 from surpassing the drive torque by the electric motor 72, stop of the rotation of the spiral tube 31 of the rotating unit 30 can be prevented.

Therefore, in the present embodiment, as described above, regulation of the flexural rigidity or the maximum bending angle of the spiral tube 31 of the rotating unit 30 and/or the second flexible tube part 25 is set. Due to this, the spiral tube 31 does not excessively bend and stop of the rotation of the spiral tube 31 is prevented.

Specifically, when the insertion portion 3 is inserted into a body cavity, the spiral tube 31 bends into various shapes according to the running shape and movability of the body cavity.

In this bent spiral tube 31, the bending of the inside of the bending spiral tube 31 is compressed for smooth rotation. In the bending of the outside, a stretching force due to pulling and a force of friction with the second flexible tube part 25 and a force of friction with a body cavity wall are generated. Thus, sufficient drive torque by the electric motor 72 is necessary.

At this time, when the bending shape of the spiral tube 31 bends with a large angle, or small radius of curvature, or three-dimensionally bends, the drive torque by the electric motor 72 necessary for rotation is needed.

Furthermore, the endoscope 2 has the configuration that regulates the flexural rigidity or the maximum bending angle larger than a reaction force as an external force received when the bending body cavity intends to keep the shape thereof according to the propulsive force when the part of the second flexible tube part 25 to which the spiral tube 31 of the rotating unit 30 provided in the insertion portion 3 is mounted advances or retreats in contact with the body cavity wall based on the rotation of the spiral tube 31 and an advancing/retreating force due to pushing and pulling of the insertion portion 3 by the user. This can prevent stop of the rotation of the spiral tube 31.

Therefore, the endoscope apparatus 1 of the present embodiment has the configuration in which, as described above, various configurations are combined and, regarding the flexural rigidity of the tube part 32 of the spiral tube 31 and/or the flexural rigidity of the second flexible tube part 25, the total flexural rigidity is set based on the structure of the part of the second flexible tube part 25 to which the spiral tube 31 is mounted based on various kinds of parameters (constituent elements based on a structure of various kinds of members) and stop of the rotation of the spiral tube 31 is prevented by the predetermined drive torque by the electric motor 72.

That is, the total flexural rigidity is set based on the structure of the part of the second flexible tube part 25 to which the spiral tube 31 is mounted through combining the configuration in which the flexural rigidity of the tube part 32 of the spiral tube 31 described in the first form or the second form is set and the configuration in which the flexural rigidity of the second flexible tube part 25 described in the first form or the second form is set. This can provide the configuration in which the spiral tube 31 does not excessively bend and stop of the rotation of the spiral tube 31 is prevented.

In the endoscope apparatus 1, by regulating the maximum bending angle of either the spiral tube 31 of the third form or the second flexible tube part 25 of the third form by the bending regulating pieces 32e or 91b to keep the spiral tube 31 or the second flexible tube part 25 from bending beyond it, excessive bending of the spiral tube 31 can be avoided and stop of the rotation of the spiral tube 31 can be prevented.

That is, in the case of using such bending regulating pieces 32e or 91b, either the spiral tube 31 or the second flexible tube part 25 may remain with a conventional configuration.

Furthermore, it is also possible to set the total flexural rigidity based on the structure of the part of the second flexible tube part 25 to which the spiral tube 31 is mounted with use of the second flexible tube part 25 with a conventional configuration and with use of only the configuration in which the flexural rigidity of the tube part 32 of the spiral tube 31 described in the first form or the second form is set.

Moreover, it is also possible to set the total flexural rigidity based on the structure of the part of the second flexible tube part 25 to which the spiral tube 31 is mounted with use of the spiral tube 31 with a conventional configuration and with use of only the configuration in which the flexural rigidity of the second flexible tube part 25 described in the first form or the second form is set.

Due to the contents described above, in the endoscope apparatus 1 that is the insertion apparatus of the present embodiment, when the insertion portion 3 is inserted into a body cavity, the rotation of the spiral tube 31, which is a driven member, does not stop even when the insertion portion 3 bends into various shapes according to the bending state, movability, and so forth, of the body cavity.

For this reason, in the endoscope apparatus 1, output power similar to conventional output power can be used as the output power of the rotational torque, or drive torque, generated by the electric motor 72, which is the drive source, to prevent stop of the rotation of the spiral tube 31, and increase in the size of the electric motor 72 is also unnecessary. Due to this, in the endoscope apparatus 1, increase in the size of the operation unit 5, in which the electric motor 72 is provided, can also be prevented and increase in the weight also does not occur.

In the endoscope apparatus 1, the need to provide a reduce or the like for increasing the rotational torque of the electric motor 72 in the operation unit 5 or the rotating unit 30 is also eliminated.

Therefore, the endoscope apparatus 1 of the present embodiment allows the spiral tube 31, which is a driven member, to smoothly rotate by the predetermined driving force by the electric motor 72, which is the drive source, and can prevent increase in the diameter of the insertion portion 3 or increase in the size and weight of the operation unit 5.

The present disclosure is not limited to only the above-described embodiment and can be carried out with various modifications in such a range as not to depart from the gist of the disclosure.

According to the present disclosure, insertion apparatus that allows a driven member to smoothly rotate by a predetermined driving force and prevents increase in the diameter of the insertion portion or increase in the size and weight of the operation unit can be implemented.

The present disclosure is not limited to the above-described embodiment and various changes, alterations, and so forth are possible in a range in which the gist of the present disclosure is not changed.

In sum, the disclosed technology is directed to an insertion apparatus comprises an insertion portion having a tubular body freely rotates around a longitudinal axis over an outer circumferential surface. The insertion portion is flexible and configured to be inserted into a body cavity. A drive source is configured to rotate the tubular body in which a part of the insertion portion includes a predetermined flexural rigidity to which the tubular body being mounted thereto. The part of the insertion portion is formed of a structure that is configured in such a manner that bending of the tubular body is not caused beyond a predetermined bending angle based on the predetermined flexural rigidity so as to avoid stop of rotation of the tubular body by a driving force of the drive source even when an external force that intends to keep a bending shape of the body cavity is received from a wall of the body cavity in contact.

A corrugated tube is incorporated in the part of the insertion portion to which the tubular body is mounted thereto. A helical tube is incorporated in the part of the insertion portion to which the tubular body is mounted thereto. A plurality of bending regulating pieces are incorporated in the part of the insertion portion to which the tubular body is mounted and configuration is made in such a manner that bending is limited to the predetermined bending angle. The tubular body is detachably attached to the outer circumferential surface of the insertion portion. The tubular body is a spiral tube having a helical-shaped fin inclined with respect to the longitudinal axis on an outer circumferential surface of the tubular body.

While various embodiments of the disclosed technology have been described above, it should be understood that they have been presented by way of example only, and not of limitation. Likewise, the various diagrams may depict an example schematic or other configuration for the disclosed technology, which is done to aid in understanding the features and functionality that can be included in the disclosed technology. The disclosed technology is not restricted to the illustrated example schematic or configurations, but the desired features can be implemented using a variety of alternative illustrations and configurations. Indeed, it will be apparent to one of skill in the art how alternative functional, logical or physical locations and configurations can be implemented to implement the desired features of the technology disclosed herein.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one”, “one or more” or the like; and adjectives such as “conventional”, “traditional”, “normal”, “standard”, “known” and 2 0 terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more”, “at least”, “but not limited to” or other like phrases in some instances shall not be read to mean that the narrower case is intended or required in instances where such broadening phrases may be absent. Additionally, the various embodiments set forth herein are described in terms of exemplary schematics, block diagrams, and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular configuration.

What is claimed is:

1. An insertion apparatus comprising:
an insertion portion includes a tubular body freely rotates around a longitudinal axis over an outer circumferential surface, the insertion portion is flexible and configured to be inserted into a body cavity; and
a drive source configured to rotate the tubular body,
wherein a part of the insertion portion includes a predetermined flexural rigidity to which the tubular body being mounted thereto, and
the part of the insertion portion is formed of a structure that is configured in such a manner that bending of the tubular body is not caused beyond a predetermined bending angle based on the predetermined flexural rigidity so as to avoid stop of rotation of the tubular body by a driving force of the drive source even when an external force that intends to keep a bending shape of the body cavity is received from a wall of the body cavity in contact.

2. The insertion apparatus of claim 1, wherein a corrugated tube is incorporated in the part of the insertion portion to which the tubular body is mounted thereto.

3. The insertion apparatus of claim 1, wherein a helical tube is incorporated in the part of the insertion portion to which the tubular body is mounted thereto.

4. The insertion apparatus of claim 1, wherein a plurality of bending regulating pieces are incorporated in the part of the insertion portion to which the tubular body is mounted and configuration is made in such a manner that bending is limited to the predetermined bending angle.

5. The insertion apparatus of claim 1, wherein the tubular body is detachably attached to the outer circumferential surface of the insertion portion.

6. The insertion apparatus of claim 1, wherein the tubular body is a spiral tube having a helical-shaped fin inclined with respect to the longitudinal axis on an outer circumferential surface of the tubular body.
